(19) United States
(12) Patent Application Publication
(10) Pub. No.: US 2006/0184209 A1
(43) Pub. Date: Aug. 17, 2006

(54) Device for brain stimulation using RF energy harvesting

(76) Inventors: Constance M. John, San Francisco, CA (US); Varghese John, San Francisco, CA (US); Martin H. Mickle, Pittsburgh, PA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/219,404

(22) Filed: Sep. 2, 2005

Related U.S. Application Data
(60) Provisional application No. 60/606,853, filed on Sep. 2, 2004.

(51) Int. Cl. A61N 1/34

(52) U.S. Cl. 607/45

(57) ABSTRACT

A device for brain stimulation using radio frequency harvesting is disclosed. The device includes a circuit implantable under a scalp of a patient, the circuit comprising a radio frequency harvesting power circuit and a stimulation circuit, and a plurality of electrodes coupled to the circuit, the plurality of electrodes providing brain stimulation to targeted areas of the brain. The electrodes may provide stimulation to targeted areas of the brain including deep brain stimulation for the treatment of Parkinson's disease and cortical stimulation for the treatment of stroke victims.

### CROSS REFERENCE TO RELATED APPLICATIONS

The present application claims priority under 35 U.S.C. 119(e) from provisional patent application Ser. No. 60/606,853, entitled “Device For Deep Brain Stimulation (DBS) Using RF Energy Harvesting”, filed on Sep. 2, 2004, the disclosure of which is herein incorporated by reference in its entirety.

### FIELD OF THE INVENTION

The present invention relates generally to systems and apparatus for providing brain stimulation and more particularly to a device for harvesting radio frequency (RF) energy that can be implanted under a human scalp to produce stimulation in different regions of the brain, including deep brain stimulation (DBS).

### BACKGROUND OF THE INVENTION

DBS is a surgical technique first used in humans over 25 years ago. DBS has been used in a wide variety of brain targets, including the thalamus, globus pallidus and the subthalamic nucleus. Diseases that have been effectively treated with DBS include movement disorders including essential tremor [Lyons K E, Pahwa R. Deep Brain Stimulation and Essential Tremor, J Clin Neurophysiol. 2004 January-February;21(1):2-5], Parkinson's disease [Byrd D L, Marks W J Jr, Starr P A. Deep brain stimulation for advanced Parkinson's disease. AORN J. 2000 September;72(3):387-90, 393-408] and dystonia [Vidailhet M. et al., Bilateral deep-brain stimulation of the globus pallidus in primary generalized dystonia. N Engl J Med. 2005 Feb. 3;352(5):459-67]. Other indications for DBS are being explored, including chronic pain, cluster headache, persistent vegatative state, epilepsy, Alzheimer's, and psychiatric disorders including obsessive-compulsive disorder and intractable depression.

Parkinson's disease (PD) is an idiopathic neurodegenerative disorder that is characterized by the presence of tremor, rigidity, akinesia or bradykinesia (slowness of movement) and postural instability. It is believed to be caused by the loss of a specific, localized population of neurons in a region of the brain called the substantia nigra. These cells normally produce dopamine, a neurotransmitter that allows brain cells to communicate with each other. These dopaminergic cells in the substantia nigra are part of an elaborate motor circuit in the brain that runs through a series of discrete brain nuclei known as the basal ganglia that control movement. It is believed that the symptoms of PD are caused by an imbalance of motor information flow through the basal ganglia.

Conventionally, a medication known as levodopa has been the mainstay of treatment for patients with Parkinson's disease. However, long-term therapy with levodopa has several well-known complications that limit the medications effectiveness and tolerability. The first of these is the development of involuntary movements known as dyskinesias. These movements can be violent at times and as or more disabling than the Parkinson's symptoms themselves. The other frequent complication is the development of “on-off” fluctuations, where patients cycle between periods of good function (the “on” period) and periods of poor function (the “off” period). These fluctuations can become very frequent, up to 7 or more cycles per day, and can cause patients to become suddenly and unpredictably “off” to the point where they cannot move.

Lesioning procedures such as pallidotomy were known to improve the motor symptoms of Parkinson's disease, presumably by disruption of the abnormal neuronal activity in the motor circuitry of the basal ganglia. The discovery that MPTP (1-methyl-4-phenyl-1,2,3,6-tetrahydropyridine) produced a Parkinsonian-like state in non-human primates allowed electrophysiologic study of this phenomenon by numerous investigators. The discovery that high frequency stimulation could mimic the effect of lesioning led to the use of DBS for PD in humans in the early 1990's. DBS was found to improve all of the cardinal symptoms of Parkinson's disease while allowing the patient to decrease or sometimes even eliminate the amount of levodopa medication, therefore decreasing both dyskinesia and “on-off” fluctuations.

DBS is currently the surgical treatment of choice for medically refractory Parkinson's disease. Two brain targets have been found to provide clinical benefit when chronically stimulated; the subthalamic nucleus (STN) and the internal segment of the globus pallidus (GPi). In a recent prospective, double-blinded cross-over study involving 96 patients with STN DBS and 38 patients with GPi DBS, the STN group reported an improvement in the percentage of time spent during the day with good mobility and without dyskinesia from 27% to 74%. The GPi group also reported a significant improvement, from 28% to 64%.

Although the mechanism of action is not fully understood it is thought to act by either depolarization blockade, synaptic inhibition, synaptic depression, or stimulation-induced modulation of pathologic network activity [McIntyre C C, Savasta M, Walter B L, Vitek J L. How does deep brain stimulation work? Present understanding and future questions. J Clin Neurophysiol. 2004 January-February;21(1):40-50]. It is believed that DBS acts somehow to suppress the neuronal activity by the stimulation of the region of the brain immediately adjacent to the electrode. This hypothesis seems to be supported by the fact that lesioning a specific structure in the brain has the same clinical effect as stimulating that same structure at high (greater than 100-150 Hz) frequency. In fact, DBS has largely replaced the older lesioning procedures (such as pallidotomy and thalamotomy) that used to be the mainstay of surgical treatment for movement disorders such as Parkinson's disease. The high frequency stimulation may act to hyperpolarize immediately adjacent neurons such that they become incapable of producing normal action potentials. An alternative hypothesis is that DBS may be altering more distant structures or even fibers from far removed nerve cells that are passing through or near the area of stimulation. Whatever the mechanism of action, DBS has a distinct advantage over the older lesioning techniques because it is an adjustable therapy and does not involve destruction of the patient's brain tissue.

Prior art DBS devices have several limitations that can lead to adverse effects including infection, cutaneous erosion, and lead breaking or disconnection [Temel Y, Ackermans L, Celik H, Spincemaille G H, Van Der Linden C, Walenkamp G H, Van De Kar T, Visser-Vandewalle V. Management of hardware infections following deep brain stimulation. Acta Neurochir (Wien) 2004;146(4):355-61; Putzke J D, Wharen R E, Jr., Wszolek Z K, Turk M F, Strongosky A J, Uitti R J. Thalamic deep brain stimulation for tremor-predominant Parkinson's disease. Parkinsonism Relat Disord 2003;10(2):81-8.; Umemura A, Jaggi J L, Hurtig H I, Siderowf A D, Colcher A, Stern M B, Baltuch G H. Deep brain stimulation for movement disorders: morbidity and mortality in 109 patients. J Neurosurg 2003;98(4):779-84; Hariz M I. Complications of deep brain stimulation surgery. Mov Disord 2002;17(Suppl 3):S162-6]. One study found that 27% of 66 patients with implanted DBS devices had hardware problems [Kondziolka D, Whiting D, Germanwala A, Oh M. Hardware-related complications after placement of thalamic deep brain stimulator systems. Stereotact Funct Neurosurg 2002;79(3-4):228-33. This relatively high incidence of hardware problems is similar to the results of a study where 20 (25.3%) of 79 patients who received 124 permanent DBS electrode implants had 26 hardware-related complications [Oh M Y, Abosch A, Kim S H, Lang A E, Lozano A M. Long-term hardware-related complications of deep brain stimulation. Neurosurgery 2002;50(6):1268-74; discussion 1274-6]. In addition, intracranial electrode implantation can induce a hematoma or contusion. Nonetheless, most authors agree that the benefit to risk ratio of DBS is favorable.

A prior art DBS device is shown in FIG. 1 and includes an electrode 100 disposed in a targeted area of the brain. The electrode is coupled to a lead 110 held in place at the top of the skull by a securement device 120. The lead 110 is coupled to a neurostimulator 130 powered by a battery-powered pulse generator 140 by means of a lead 150. The lead 150, which averages about 15 inches in length, is implanted under the scalp and traverses the length of the patient's neck to the chest where the neurostimulator 130 and battery 140 are implanted.

The pulse generator 140 is typically placed underneath the skin just below the collar bone and is capable of stimulating at one or any combination of the four contacts present on the end of the electrode 110 in the brain. The parameters of the stimulating current (voltage, frequency, pulse width) can also be selected by the treating physician or health care worker. The pulse generator 140 is programmed through the skin via a telemetry device that allows the practitioner to select the desired stimulation parameters and also perform diagnostic tests on the device.

Implantation of the conventional DBS device is costly as for implantation of a single electrode in the brain for treatment of one side of the body the procedure requires three incisions; one on the top of the head, one behind the ear and the third just below the collarbone where the leads are connected. The implantation of the electrode 110 and the implantable pulse generator 140 is sometimes performed on different days. The incisions can be prone to infection in the immediate postoperative period. In some elderly patients with thin skin, the pulse generator 140 or wire can erode through the skin and become exposed to potential contamination. Infection or erosion often results in the need to remove the entire device, as antibiotic treatment alone in this setting rarely will clear the infection adequately. The lead 150 restricts the patient's mobility in the neck region and may break. Furthermore, the battery 140 must be replaced every three to five years. Additional drawbacks of the DBS device include the risk of erosion of the leads or hardware, infection, and magnetic sensitivity.

A prior art deep brain stimulation system is disclosed in U.S. Pat. No. 6,920,359 entitled “Deep Brian Stimulation System for the Treatment of Parkinson's Disease or Other Disorders”. The DBS system includes a small, implantable pulse generator implanted directly in the cranium of the patient, thereby eliminating the long lead wires conventionally used. The disclosed system does not provide for the harvesting of energy to power the pulse generator.

As noted in Table 1, there are several current and potential indications for deep brain stimulation.

Known systems for providing electrical stimulus to the motor cortex of the brain, such as the Northstar Stroke Recovery Treatment System available from Northstar Neuroscience, Inc., also include an implantable pulse generator implanted in the pectoral area of a patient. A cortical stimulation lead includes an electrode connected to the implantable pulse generator which is used to deliver stimulation to the cortex. The electrode is placed on top of the dura and coupled to the implantable pulse generator by means of a lead which traverses the length of the patient's neck to the patient's pectoral area.

Motor cortex stimulation (MCS) is a process involving the application of stimulation signals to the motor cortex in the brain of a patient during physical rehabilitation of the disabled region of the body. The MCS system includes a pulse generator connected to a strip electrode that is surgically implanted over a portion of only the motor cortex (precentral gyrus). Because MCS involves the application of stimulation signals to surface regions of the brain rather than deep neural structures, electrode implantation procedures for MCS are significantly less invasive and time consuming than those for DBS. The current evaluation of MCS is for stroke. Stroke-related disabilities affect more than 200,000 people in the U.S. each year. Good results have been reported in MCS treatment of stroke victims. With a MCS device, a stamp-sized electrode is placed on the surface of the brain. This is attached to a wire that goes through the neck to an implantable pulse generator in the pectoral area.

Neurostimulation and responsive neurostimulation (RNS) are also being tested for the treatment of medically refractory epilepsy. In treating epilepsy, the RNS system can be designed to detect abnormal electrical activity in the brain and respond by delivering electrical stimulation to normalize brain activity before the patient experiences seizure symptoms. For either neurostimulation or RNS for treatment of epilepsy the electrode or electrodes of the device deliver a short train of electrical pulses to the brain near the patient's seizure focus.

In order to obviate the need for long leads and batteries, attempts have been made in the prior art to transmit energy through space from a base station to a remote station. One such system is disclosed in U.S. Pat. No. 6,289,237 entitled “Apparatus for Energizing a Remote Station and Related Method”. The base station transmits energy which may be RF power, light, acoustic, magnetic or other suitable forms of space transmitted or “radiant” energy to the remote station. Within the remote station, the received energy is converted into DC power which serves to operate the remote station. The source of power for the remote station is the base station and, therefore, there is no need for the remote station to carry an electrical storage device such as a battery. It is suggested that this facilitates the remote station being encapsulated within a suitable protective material, such as a resinous plastic. Homopolymers, elastomers and silicon dioxide are also suggested as suitable materials for such purposes. Further, it is suggested that this facilitates miniaturization of the remote station and placing the remote station in functionally desirable locations which need not be readily accessible. The remote station, for example, could be implanted in a patient.

The use of a wireless communication link between a base station and transponders in a radio frequency identification system employing modulated back-scattered waves is also known. See Rao, An Overview of Bulk Scattered Radio Frequency Identification System (RFID) IEEE (1999). It has also been suggested to employ a silicon chip in a transponder having a change pump on voltage doubler current. Hornby, RFID Solutions for the Express Parcel and Airline Baggage Industry, Texas Instruments, Limited (Oct. 7, 1999).

For use in miniaturized electronic chip systems, an electronic article containing a microchip having at least one antenna structured to communicate with an antenna remotely disposed with respect to the microchip is disclosed in U.S. Pat. No. 6,615,074 entitled “Apparatus for Energizing a Remote Station and Related Method”. Power enhancement is achieved using a voltage doubler. The antenna of the disclosed apparatus is comparable in volume to a Smart Dust device. Smart Dust is a combination MEMS/Electronic device on the order of 1 mm×1 mm×1 mm.

What is needed therefore is a brain stimulation device that overcomes the disadvantages of the prior art brain stimulation devices. What is needed is a brain stimulation device that requires a single implantation site and surgery. What is also needed is a brain stimulation device that uses RF energy as a power source. What is further needed is a brain stimulation device that converts RF energy and stores the converted RF energy. What is also needed is a brain stimulation device that is flexible and implantable under the scalp. What is needed is a brain stimulation device that does not require leads or a pulse generator to be placed outside of the head area that are subject to disconnection or breakage. What is also needed is a brain stimulation device for electrical stimulation in the brain that is smaller and more self-contained and that does not require a pulse generator to be implanted elsewhere in the body. What is further needed is a device that is less susceptible to hardware problems or complications. What is needed is a device that has less potential for erosion through the skin. What is also needed is a device that is has a power source that does not need to be replaced.

### SUMMARY OF INVENTION

The device for brain stimulation using RF energy harvesting of the present invention overcomes the disadvantages of the prior art, fulfills the needs in the prior art, and accomplishes its various purposes by providing a brain stimulation device that harvests radio frequency energy and is implantable under the scalp. The brain stimulation device of the invention may include an electrode that penetrates into the brain to provide neurostimulation to the brain. The brain stimulation device may also include an electrode that is used to provide stimulation to the brain cortex.

In accordance with one aspect of the invention, a device for brain stimulation using radio frequency harvesting includes a circuit implantable under a scalp of a patient, the circuit comprising a radio frequency harvesting power circuit and a stimulation circuit, and a plurality of electrodes coupled to the circuit, the plurality of electrodes providing brain stimulation to targeted areas of the brain. An advantage of this system is that it may use “trickle charging” wherein the device is charged by the harvesting power circuit. Moreover, anther advantage of this invention is the power transmitter which sends power to the device can be used both to send power and to send information.

There has been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended herein.

In this respect, before explaining at least one embodiment of the invention in detail, it is to be understood that the invention is not limited in its application to the details of design and to the arrangement of components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other methods and systems for carrying out the several purposes of the present invention. It is important, therefore, that the claims be regarded as including such equivalent methods and systems insofar as they do not depart from the spirit and scope of the present invention.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 is a schematic representation of a prior art DBS device;

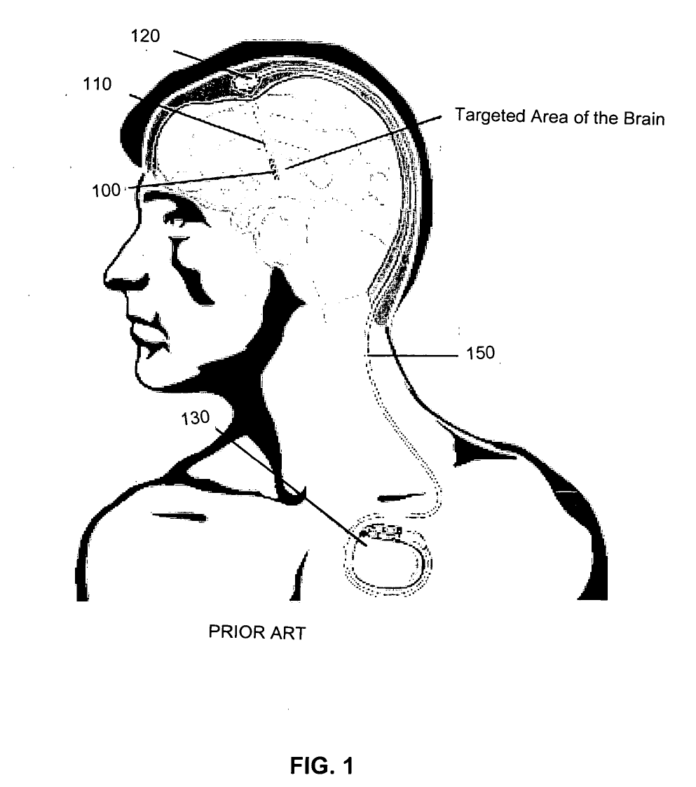

FIG. 2A is a schematic representation of a device for deep brain stimulation using RF energy harvesting in accordance with the invention;

FIG. 2B is a schematic representation of a device for cortical stimulation using RF energy harvesting in accordance with the invention;

FIG. 2C is a schematic representation of the device of FIG. 2A illustrating lead securement devices;

FIG. 2D is a schematic representation of the device of FIG. 2A illustrating an attachment means for connecting a lead wire to a circuit of the device;

FIG. 3 is a schematic representation of a stimulation circuit in accordance with the invention;

FIG. 4 is a graph showing an output enable pulse from a microcontroller of the stimulation circuit shown in FIG. 3 in accordance with the invention;

FIG. 5 is a graph showing an output signal from the microcontroller of the stimulation circuit shown in FIG. 3 applied across a resistive load in accordance with the invention.

FIG. 6 is a graph showing pulses across the resistive load;

FIG. 7 is a schematic representation of an external programming circuit in accordance with the invention;

FIG. 8A is a schematic representation of an external power circuit inductively coupled to a power circuit in accordance with the invention;

FIG. 8B is a schematic representation of an alternative embodiment of the external power circuit non-inductively coupled to the power circuit in accordance with the invention;

FIG. 9 is an illustration of a PCB layout of the stimulating circuit in accordance with the invention;

FIG. 10 is an illustration of the external power circuit in accordance with the invention/

FIG. 11 is an oscilloscope screen showing the output voltage from an oscillator of the external power circuit in accordance with the invention;

FIG. 12 is an oscilloscope screen showing a voltage across a primary coil series resistance of the external power circuit in accordance with the invention;

FIG. 13 is a graph showing the effect of skin disposed between the primary coil and a secondary coil of the stimulating circuit in accordance with the invention;

FIG. 14 is a graph showing the effect of freezing the thawing the skin in accordance with the invention;

FIG. 15 is a graph of the output voltage over time in accordance with the invention; and

FIG. 16 is a pictoral representation of a model having the stimulation circuit implanted in a scalp and the external powering circuit disposed in a hat in accordance with the invention.

### DETAILED DESCRIPTION OF THE INVENTION

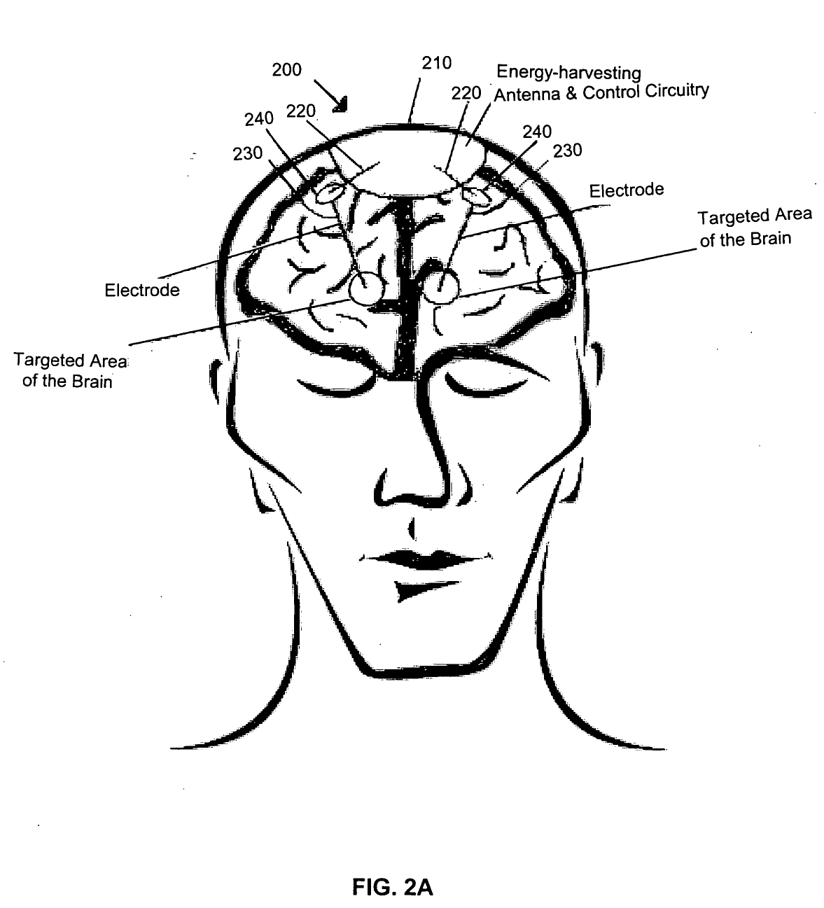

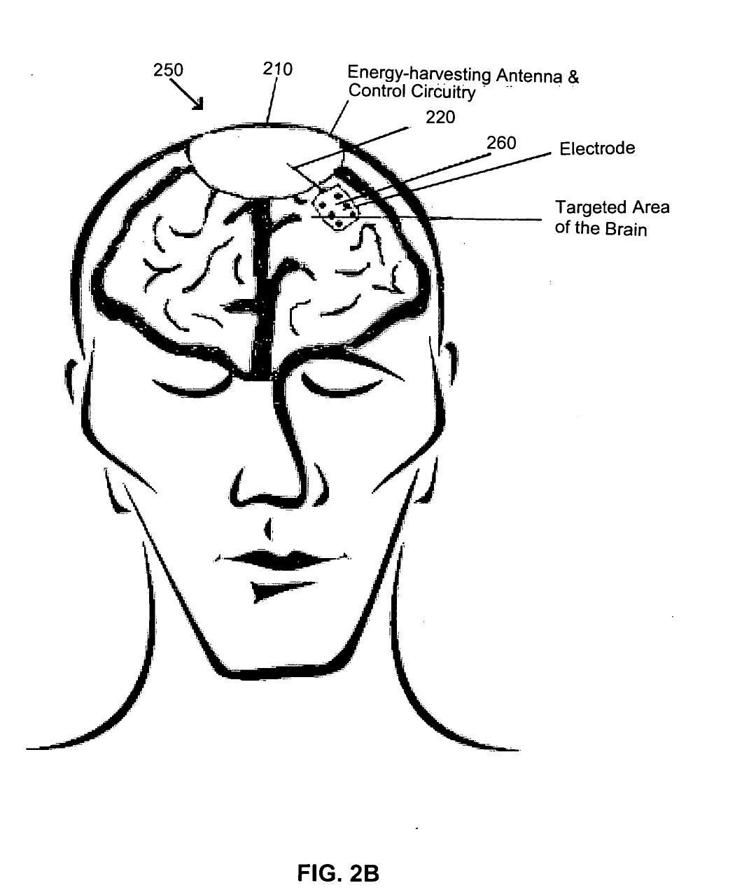

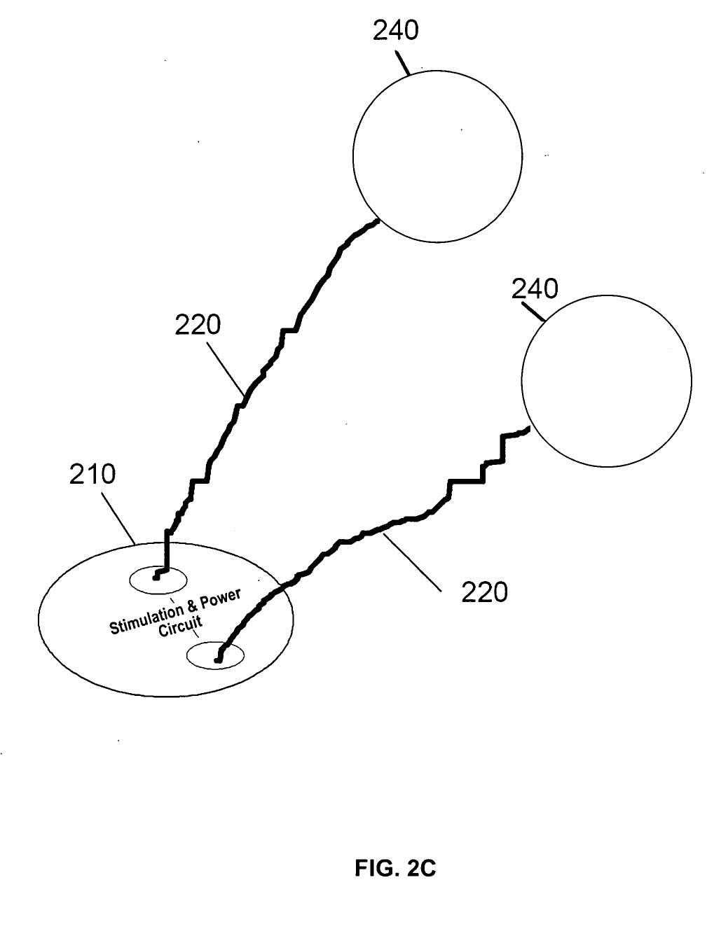

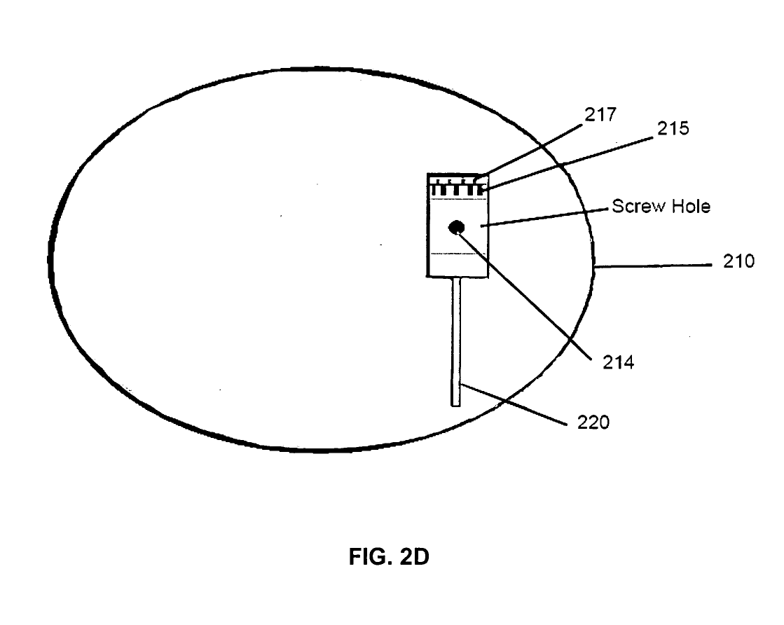

A device for deep brain stimulation using RF energy harvesting 200 of he invention is shown implanted under a human scalp in FIG. 2A. A flexible, implantable disc-shaped portion 210 having a diameter of about 6 cm and a thickness of between 3 and 4 mm may be formed of a biocompatible material and include circuitry as further described herein. Lead wires 220 may lead from the circuitry and be coupled to electrodes 230 disposed in targeted areas of the brain. Electrodes 230 may include conventional electrodes used for DBS. Neurostimulation lead securement devices 240 including burr hole caps may serve to secure the lead wires 220 to the electrodes. The circuitry may be operable to harvest and store RF energy, control the operation of the device 200 and provide neurostimulation pulses and signals to the targeted areas of the brain.

A device for cortical brain stimulation using RF energy harvesting 250 of the invention is shown in FIG. 2B. The flexible, implantable disc-shaped portion 210 is shown implanted under the scalp. Lead wires 270 may lead from the circuitry of the disc-shaped portion 260 and be coupled to electrodes 280 disposed on the cortical dura.

With reference to FIGS. 2C and 2D, lead securement devices 240 are shown. Lead securement devices 240 may include StimLoc devices available from ign. Lead securement devices 240 may minimize dislodgment of lead wires 220.

Lead wires 220 may be coupled to the circuitry of the disc-shaped portion 210 by means of connectors 215. Connectors 215 may include a plurality of male contacts 217 for providing electrical contact to corresponding female contacts of the circuitry (not shown). A screw hole 219 may be formed in the connector 215 for securing the connectors 215 to the disc-shaped portion 210 and for securing the disc-shaped portion 210 to the skull of the patient.

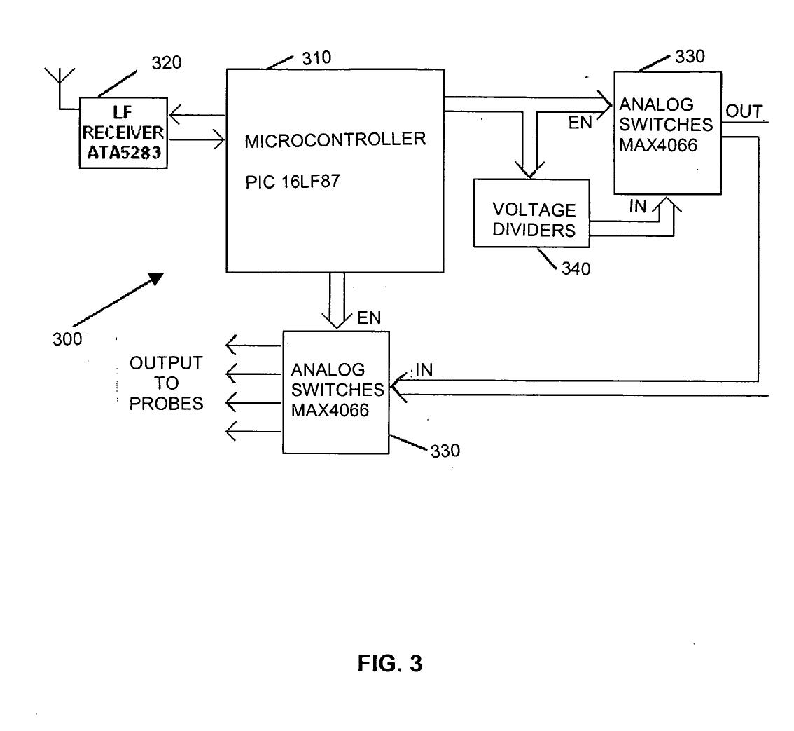

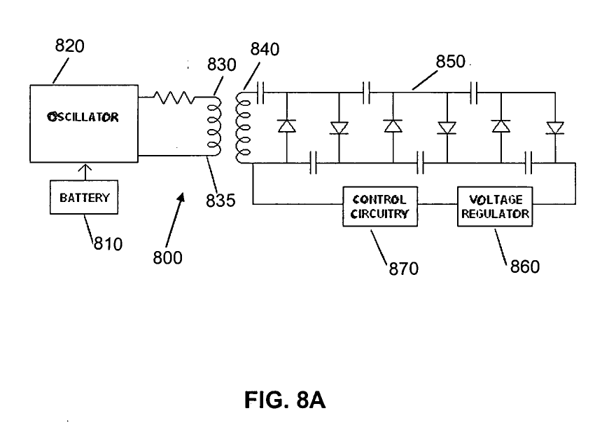

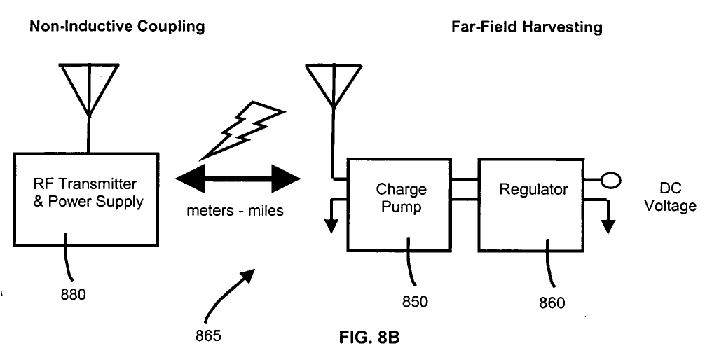

The circuitry may include a stimulation circuit 300 as shown in FIG. 3 and a portion of the power circuit as shown in FIG. 8A. The stimulation circuit 300 may include a circuit printed onto the disc-shaped portion 210. For purposes of illustration, the stimulation circuit 300 may be modeled using discreet components. The stimulation circuit 300 may include a PIC microcontroller 310 such as the PIC16LF87. The microcontroller 310 may manage the internal stimulation circuitry. A low frequency receiver chip 320 such as the ATA5283 may be coupled to the microcontroller 310 and may convert RF communications into programming commands which the microcontroller 310 interprets. An array of analog switches 330 such as the MAX4066 may be coupled to the microcontroller 310 and connect to voltage dividers 340 to output stimulation locations. Analog switches 330 may be coupled to electrodes 230 (FIG. 2A) and 260 (FIG. 2B).

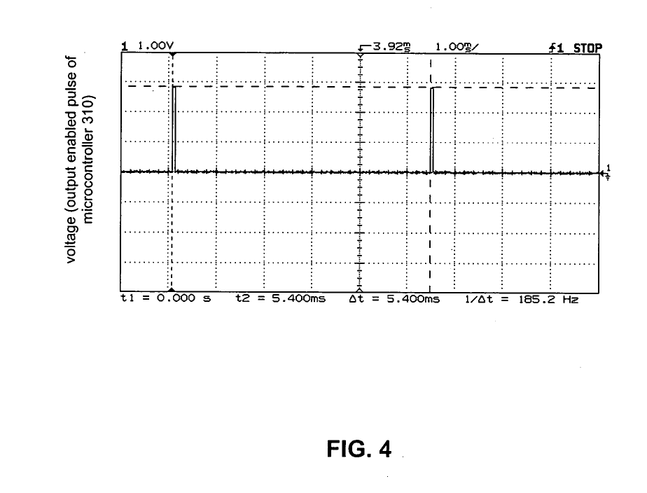

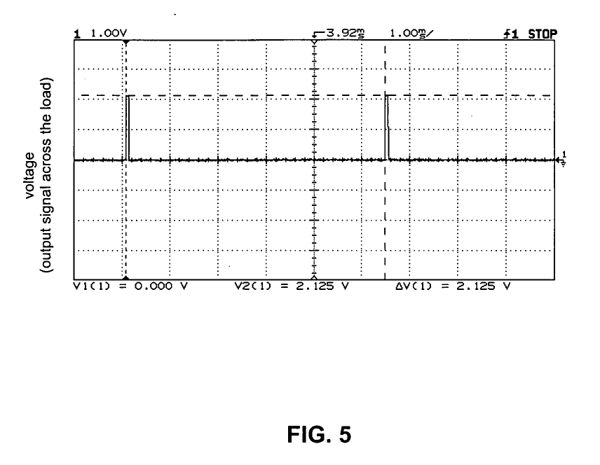

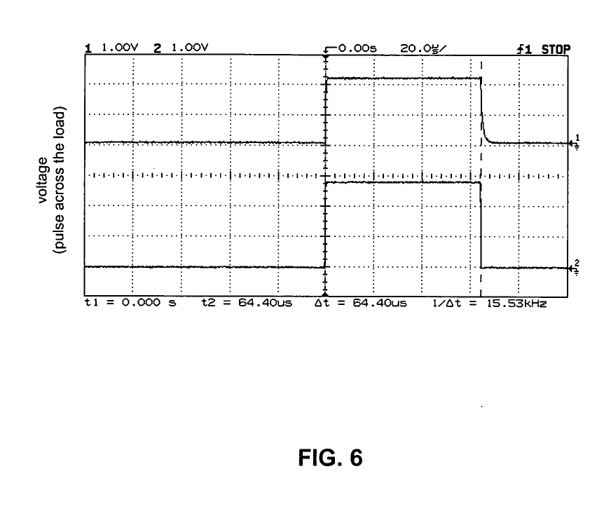

According to internal parameters which can be modified via an external RF programming signal, the microcontroller 310 may control analog switch states to determine a voltage applied to any combination of four output locations including four output locations on electrodes 230 and 260. The maximum possible voltage is determined by the supply voltage to the circuit 300. A pulsing frequency, nominally 185 Hz, can be adjusted slightly as well as whether a stimulation pulse is applied or not. In order to conserve energy, the microcontroller 310 may enter a standby mode for 4 ms between pulses, greatly reducing power consumption. The microcontroller 310 may be operated with an internal clock frequency of 125 KHz, giving an efficient tradeoff between power conservation and proper functionality. This clock frequency allows pulse durations in increments of 32 micro-seconds. The output pulse duration can be adjusted between ˜60 and ˜180 microseconds. With reference to FIG. 4, FIG. 5 and FIG. 6, the frequency output, varied voltage output and pulse duration of the microcontroller 310 are shown respectively.

Every pulsing cycle, the programming input from the low frequency receiver chip 320 may be checked. If a programming signal is present, an input code may be read sequentially and the specified parameter adjusted to a new value, after which the program continues its pulsing routine.

The low frequency receiver chip 320 used for receiving external programming commands uses an amplitude shift keying (ASK) protocol. The state of a 125 KHz signal being received determines the output voltage of the low frequency receiver chip 320: on-high, off-low. While waiting for a signal, the low frequency receiver chip 320 may remain in standby mode, conserving power. Upon the presence of a programming signal, the low frequency receiver chip 320 may wake up and send the coded data to the microcontroller 310, after which the microcontroller 310 may tell the low frequency receiver chip 320 to enter standby mode again. The programming signal may include a preliminary “on” time to wake up the low frequency receiver chip 320, a 4-bit header, a 3-bit parameter identifier, and a 4-bit data value. Each bit time is 2 milliseconds, allowing enough time for the microcontroller 310 to process the bit reception before the next bit arrives. An antenna attached to a coil input of the low frequency receiver chip 320 may be a short wire having a strong programming signal.

Eight analog switches 330 may be used to control the output pulsing. Four switches 330 may determine a path of the selected voltage to the four possible output locations. Each of these may be controlled by one of the microcontroller outputs, which are in turn enabled or disabled depending on the internal variable for output locations. The inputs of the four switches 330 may be attached to the outputs of the other four switches 330. The inputs of these four switches 330 may all be attached to different voltage dividers 340, providing four different voltage levels, ranging from three quarters of the supply voltage to the supply voltage maximum of 3V. Each switch 330 may be controlled by an individual microcontroller signal, which also drives the voltage divider 340 for its particular switch 330. For every pulse, only one of these four microcontroller signals is active, enabling the voltage from its divider 340 to be sent to the output switches 330 and ultimately to the electrodes 230 and 260. The use of static voltage dividers 340 to provide output voltage scaling may minimize power consumption. In an alternative embodiment of the invention, a custom digital-analog converter could be used to allow for a higher range of stimulation voltages.

When tested for power consumption, a ˜1 Ω resistor was put in series with a powering circuitry described herein. The voltage measured across the resistor while in operation was approximately 17 μV, implying that the DC current required is ˜17 μA. At a supply voltage of 3 V, this equates to a power consumption of 51 μW. If operated for 24 hours, the implant would consume a little over 4.4 Joules/day. Typical parameters of a stimulation signal provided for Parkinson's disease are a series of pulses of 120 micro-second duration, 2.5 volts in strength at a repetition rate of 185 pulses per second. Assuming these typical parameters, there are:
185 pulses/second*60 seconds/minute*60 minutes/hour*24 hours/day=1.5984*10⁷ pulses/day.
With pulse duration of 120 micro seconds, this gives a total energy application duration of 1.5984*10⁷*120*10^{6=1918.08} seconds. With 2.5 volts and 50 micro amps=120 micro watts, the total energy required for stimulation is 120*10⁶ watts*1918.08 seconds=0.2302 joules per day. As disclosed herein, energy harvesting by the power circuit is on the order of 12-15 joules per day and the stimulation energy required is more than adequately provided by the power circuit.

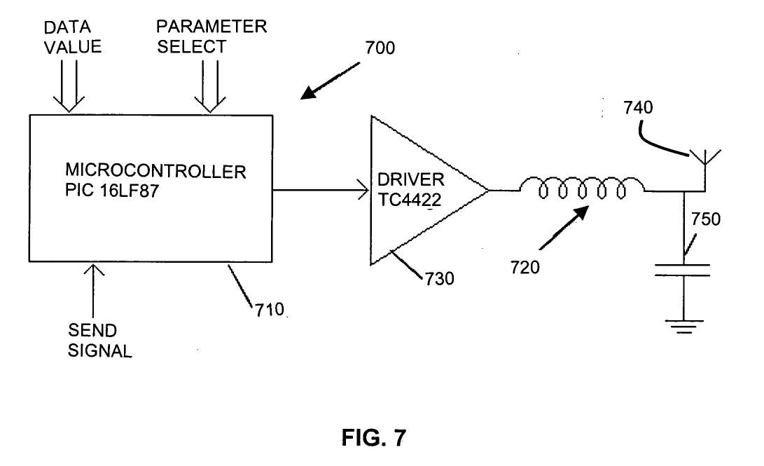

An external programmer circuit 700 may include a microcontroller 710 including a PIC16LF87, an inductor/capacitor (LC) oscillating circuit 720 (125 KHz), and an intermediate MOSFET driver 730 including a TC4422 as shown in FIG. 7. The MOSFET driver 730 may supply enough energy for driving the LC circuit 720. When a programming signal is to be sent, a button (not shown) may be pressed, telling the microcontroller 710 to read its inputs and stimulate the MOSFET driver 730 to oscillate the LC circuit 720 according to a communication protocol. Input voltages may be controlled by simple switches. Four switches may dictate the value to be sent, while five switches may dictate which parameter is to be changed. Only one of these switches should be on at one time. A Phidget RFID antenna 740 designed for 125 KHz may be attached to the high voltage side of a capacitor 750 of the LC circuit 720 for sending the programming signal. The circuit 700 may be powered via a 12-Volt wall supply. The 12 V drives the MOSFET driver 730 and is regulated to 5 V for the switches and microcontroller 710.

An external powering circuit 800 may include a battery 810 for powering an oscillator 820 which drives a transformer-like setup 830 as shown in FIG. 8A. The coils 835 on one side of the transformer 830 may be disposed in a cap worn on the head of a patient, a headband worn on the head of the patient, or on a headboard of a bed in which the patient lies. The coils 840 on the other side of the transformer 830 may be coupled to the stimulation circuit 300 and may be disposed proximate the coils 835. An AC signal coming from coil 840 may be amplified and rectified through a charge pump 850 having three stages, after which a voltage may be clamped with a regulator 860 to prevent spiking. A control circuit 870 may control operation of the voltage regulator 860.

The oscillator 820 may include an LTC6900. This oscillator 820 produces a 50% duty cycle square wave to drive the primary coil 835 of the transformer 830 and requires only a potentiometer for adjusting the frequency. The charge pump 850 may be a Cockroft Walton voltage multiplier, utilizing a ladder of diodes and capacitors to rectify and amplify the signal. The amplification depends on the number of stages used. Three stages have been found to be enough for a substantial voltage multiplication across a load of 200 KΩ. The capacitors may be 0.1-μF each and the diodes may include BAT54SW surface mount diodes with a forward voltage drop of ˜0.24-V. The regulator 860 may include an LT1521-3, which clamps a higher input voltage to 3 V.

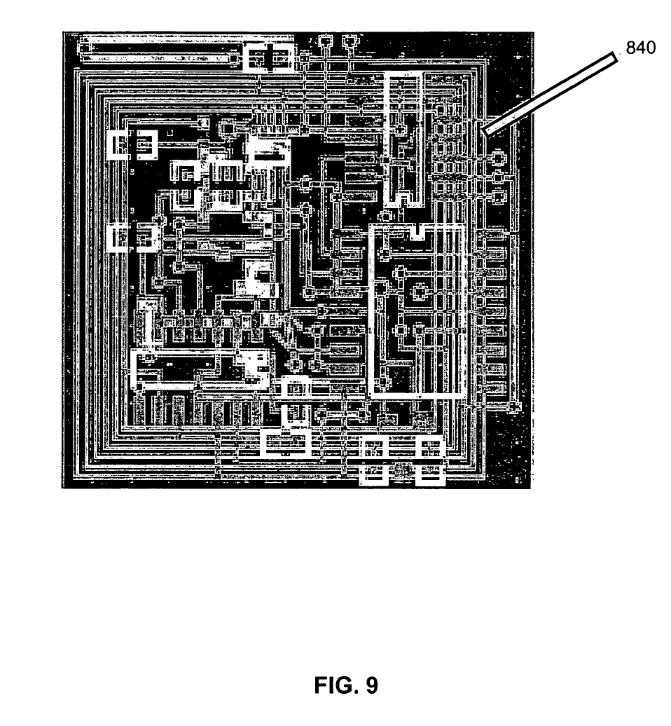

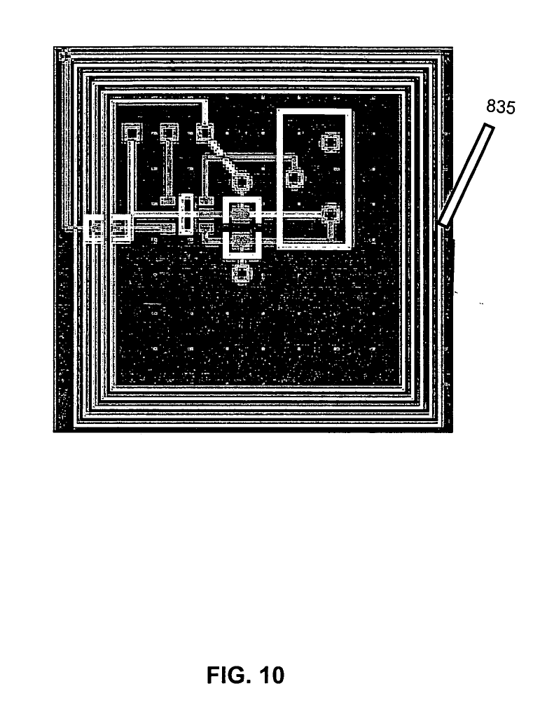

Previous empirical testing showed square coils (both primary 835 and secondary 840), 1 in.×1 in., with 5 turns each are effective for transferring enough energy to power the stimulation circuit 300. Coils 840 are shown in PCB layout in FIG. 9 and coils 835 are shown in PCB layout in FIG. 10.

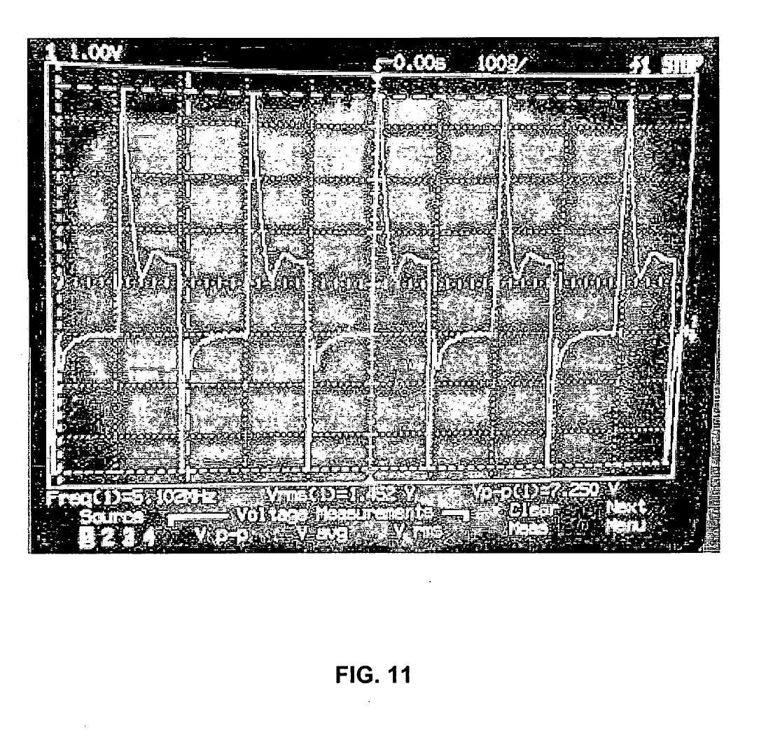

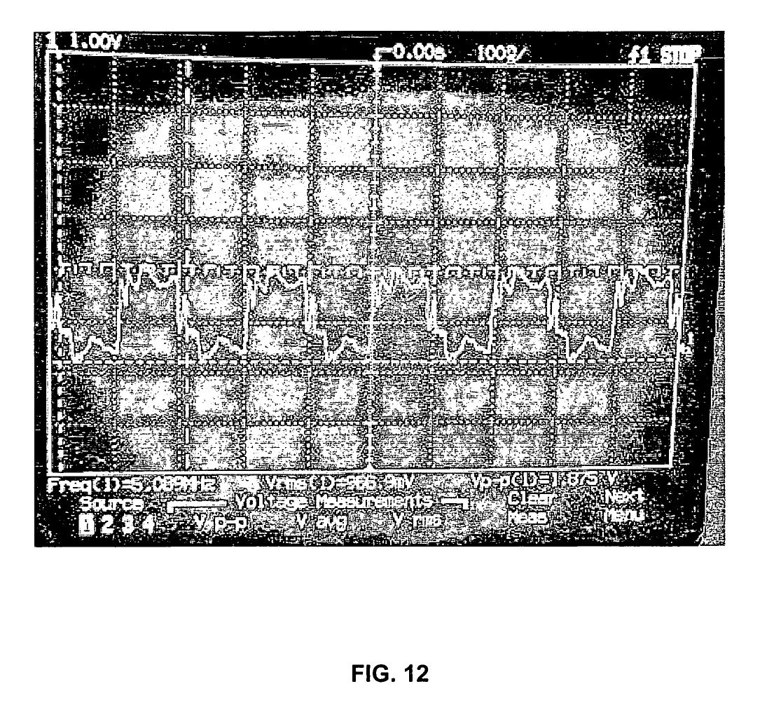

The optimal frequency depends on the dielectric and distance between coils 835 and 840. Frequencies in the range of 2 MHz to 15 MHz may be used. The oscillator 820 can be powered with 3 AAA batteries (4.5 V). In examining the actual signal through the primary coil 835, the voltage waveforms in FIG. 11 and FIG. 12 were obtained. FIG. 11 shows the output voltage from the oscillator 820. FIG. 12 shows the voltage across the primary coil series resistance, from which the RMS current is calculated to be 29.36 mA_{RMS}.

The embodiment described above provides for near field harvesting and includes inductive coupling between coils 835 and 840. With reference to FIG. 8B and in an alternative embodiment of the invention, the power circuit 865 for powering the stimulation circuit 300 may be non-inductively coupled to an external source of RF energy 880. In this far field embodiment, the power circuit 865 may be disposed in a wrist band worn by the patient, in a room transmitter or in a transmitter disposed in a building occupied by the patient. In yet another alternative embodiment of the invention, the power circuit 865 may harvest ambient RF energy such as energy transmitted in space by using an inherently tuned antenna as described in U.S. Pat. No. 6,856,291, the description of which is incorporated by reference in its entirety herein. Furthermore, a rechargeable battery or other storage device (not shown) may be employed to store harvested energy. “Non-inductive” as described herein being directed RF.

To demonstrate the effectiveness of the powering and programming schemes through tissue, the device 200 was tested through swine skin. Clear tape was used to cover the conductive surfaces on the primary coil 835 and the secondary coil 840 to prevent interaction with the moisture on the skin. This tape had negligible effect on the inductive coupling.

Three different tests were performed, each following the same procedure. At a certain separation, the voltage powering the primary coil oscillator 820 was adjusted and the maximum output voltage from the secondary coil voltage regulator 860 was measured. The primary coil oscillator voltage started at 5 V and was decreased in increments of 0.1 V until the maximum regulator output voltage had reached a steady minimum.

The first test was performed with no skin between the transformer coils 835 and 840. Data was acquired at separations of 5 mm, 7 mm, and 10 mm, values chosen based on the common range of human scalp thickness. The second test used fresh swine skin of thicknesses 5 mm and 7 mm between the transformer coils 835 and 840. The test was interrupted, preventing the testing of 10 mm thick skin. The third test used the same pieces of swine skin, 5, 7, and 10 mm thick, after they had been frozen and thawed.

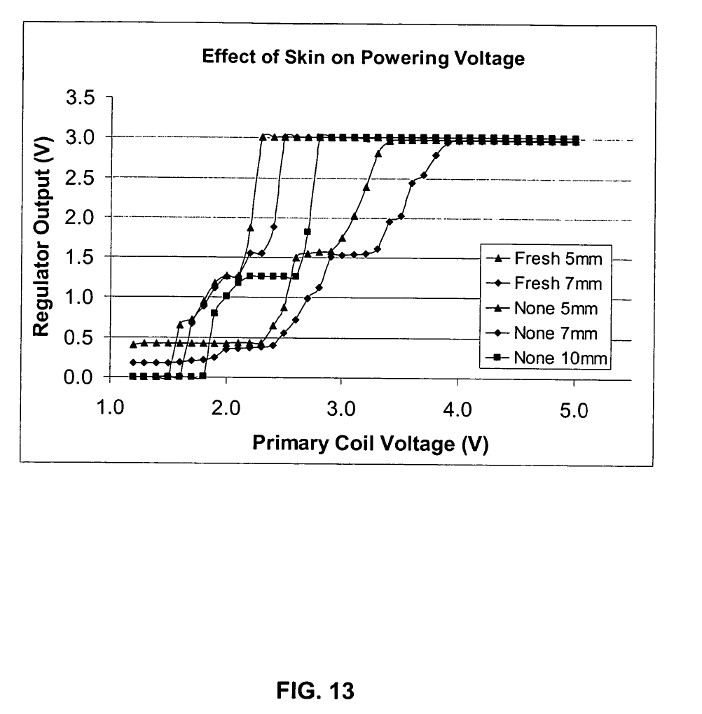

FIG. 13 shows the results from the first two tests for comparison purposes. The presence of the skin reduced the inductive coupling between the coils, and hence the possible maximum output voltage in the range of 1.5-3.8 V. At 4.0 V and above, the maximum output voltage of ˜3 V is obtainable even with the presence of the skin.

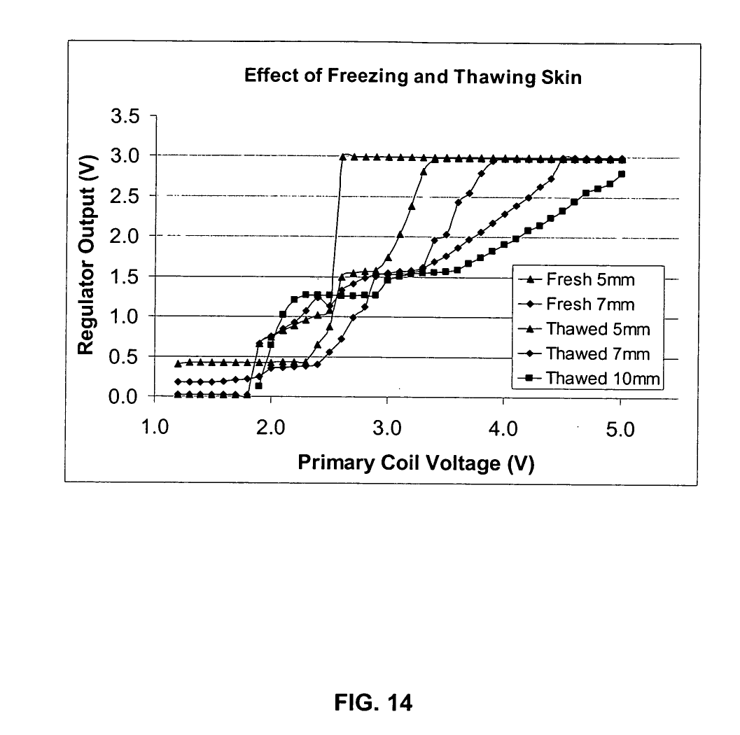

The same pieces of skin were tested a second time due to interruption of the first test. However, they had all been frozen and thawed in the interim, affecting the results slightly. FIG. 14 shows the effect that the freezing and thawing of the skin had on the energy transfer of the transformer coils. Both the 7- and 10-mm thick pieces of skin reduced the inductive coupling, but the 5-mm skin actually improved in performance. This may be due to the presence of a layer of fat in both the 7- and 10-mm pieces that is absent in the 5-mm piece.

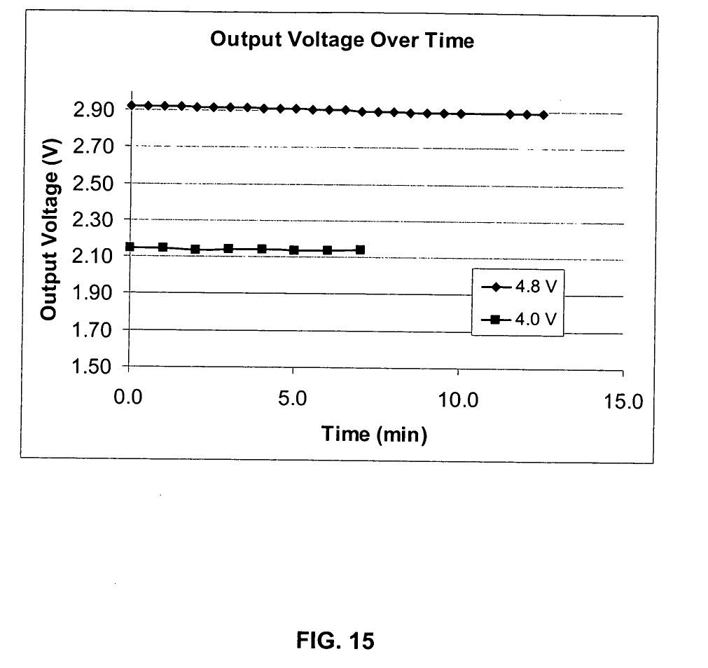

Another test was performed to find the effect of the skin over time. The stimulus for this test was the degrading performance of the 10-mm thick skin over time during the interrupted test mentioned above. For this test, the 7-mm thick piece of skin was used between the primary and secondary coil. The frequency was adjusted to produce a maximum output voltage, which was measured successively over a period of time. The results shown in FIG. 15 support the fact that performance does not degrade over time. The slight drop in output voltage is likely due to the mechanical nature of the frequency-tuning potentiometer. Notice that the output voltage reaches a steady value and remains constant after that point.

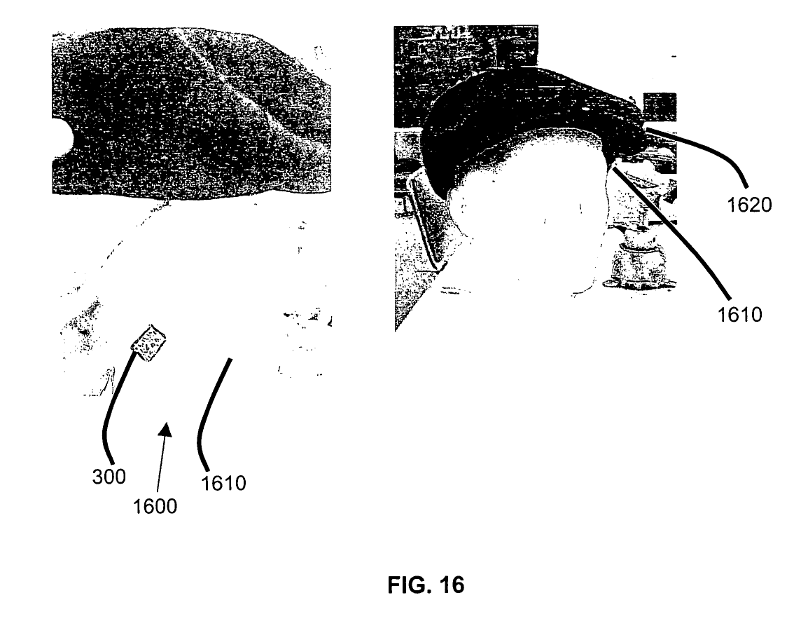

In order to demonstrate the concept and functionality of the device 200, a model 1600 was created as shown in FIG. 16. The stimulation circuit 300 was put on top of a Styrofoam head 1610 with wires running down through the bottom for power- and pulse-monitoring purposes. An ABS Plastic cap (not shown) was made to simulate the head's scalp, covering the stimulation circuit 300. The primary powering coil 835 with the batteries 810 was secured in a hat 1620 over the position of the stimulation circuit 300 to provide for near field inductive coupling.

The device 200 was tested on a cadaver head to show that a signal may be generated through the scalp and to demonstrate the programmability of the device 200 during stimulation. First, an incision was made in the scalp of the cadaver head. The secondary coil 840 was inserted and placed on the skull and the incision was sewn, leaving the lead wires 220 of the circuit exposed. Six wires were used on the implanted circuitry, four representing the electrodes 230, which were connected to an oscilloscope and two wires for power and ground. The primary coil 835 was then taped on the scalp directly on top of the implanted circuitry and connected to a power supply.

The experiment began by demonstrating the programmability of the stimulation circuit 300. Four parameters were varied and displayed on the oscilloscope; pulse width (60, 120 and 180 micro seconds), amplitude (2.34V, 2.75V, 2.94V, 3.13 V), frequency (191 and 194 Hz) and the shifting from one stimulating probe to another (i.e. probe 1 to probe 2 or probe 1 to all four probes). A 10K OHM resistor was used to represent the brain resistance, however this resistance is higher than the resistance for the brain (900 to 1100 Ohms), but a 10 k Ohm resistor was used to ensure there was enough power.

Next, several voltages were tested to determine the output source voltage of the power circuit 865. Initially, the power supply connected to the primary coil 835 was set at 5 V and was decremented by 0.1 V to 1.2 V. The voltage on the secondary coil 840 was clamped so as not to exceed 3V. As the voltage decreased on the power supply, the output voltage on the secondary coil 840 was steady at 3 V until it declined around 2.2 V. Once the voltage decreased from 3V, a potentiometer was adjusted to obtain the maximum voltage. The data obtained shows the when the voltage drops, the amplitude voltage and frequency drop off as well.

The device for brain stimulation using RF harvesting of the present invention provides a brain stimulation device that requires a single implantation site and surgery to thereby reduce both the cost and trauma to the patient of the implantation procedure. The brain stimulation device further uses RF energy as a power source to eliminate the need for a battery implanted in the pectoral area of the patient. The brain stimulation device further converts RF energy and stores the converted RF energy for use in stimulation of targeted brain areas. The brain stimulation device is flexible and implantable under the scalp to minimize discomfort for the patient.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

TABLE 1

| | | |
|---|---|---|
| Indication | Prevalence | Reference for Prevalence |

FDA Approved Indications

| | | |
|---|---|---|
| Essential tremor | 1.5-3% population | Am J Med 115:134-42, |
| (1997) | | 2003 |
| Parkinson's (2002) | 1% of population > 50 | |
| Dystonia (2004) | ˜150,000 in US | Mov Disord 3:188-94, |
| | | 1988 |

Current Clinical Trials

| | | |
|---|---|---|
| Obsessive-compulsive | 2-3% of population | J Clin Psych 53 Suppl: |
| disorder | | 4-10, 1992 |
| Tourette's syndrome | ˜1-2% children | J Psychosom Res. |
| | | 55:3-6, 2003; Can J |
| | | Neurol Sci Suppl |
| | | 1:S64-71, 2003 |
| Intractable epilepsy | ˜100,000 in US | Neurology 56: |
| | | 1445-52, 2001; Rev |
| | | Neurol (Paris); 160 Spec |
| | | No 1:5S31-5, 2004 |
| Intractable depression | 1,000,000 | Psychiatr Clin North Am |
| | | 19:179-200, 1996; |
| | | http://www.mhsource. |
| | | com/depconsult/june |
| | | 2004.jhtml?_requestid = |
| | | 605984 |

We claim:

1. A system for brain stimulation using radio frequency harvesting comprising:
a device implantable under a scalp of a patient, the device comprising a radio frequency harvesting power circuit and a stimulation circuit; and
at least one electrode coupled to the stimulation circuit, the at least one electrode providing brain stimulation to targeted areas of the brain.

2. The system of claim 1, wherein the device is fabricated from a biocompatible substrate.

3. The system of claim 1, wherein the device is flexible and conformable to a shape of the scalp.

4. The system of claim 1, wherein the power circuit comprises a charge pump inductively coupled to a primary coil of an external power circuit.

5. The system of claim 1, wherein the power circuit comprises an inherently tuned antenna for harvesting energy transmitted in space.

6. The system of claim 1, wherein the at least one electrode provides deep brain stimulation.

7. The system of claim 1, wherein the at least one electrode provides cortical stimulation.

8. The system of claim 1, further comprising a programming circuit operable to control the stimulation circuit.

9. The system of claim 8, wherein the programming circuit is operable to control a stimulation circuit voltage output.

10. The system of claim 8, wherein the programming circuit is operable to control a stimulation circuit output pulse width.

11. The system of claim 8, wherein the programming circuit is operable to control a stimulation circuit output frequency.

12. The system of claim 1, further comprising an energy storage device coupled to the device.

13. A system for brain stimulation comprising:
a device implantable under a scalp of a patient, the device comprising a coupled power circuit and a stimulation circuit; and
at least one electrode coupled to the stimulation circuit, the at least one electrode providing brain stimulation to targeted areas of the brain.

14. The system of claim 13, wherein the coupled power circuit is inductively coupled.

15. The system of claim 13, wherein the coupled power circuit is non-inductively coupled.

16. A system for brain stimulation comprising:
a device implantable under a scalp of a patient, the device comprising a power circuit powered by ambient radio frequency energy and a stimulation circuit; and
at least one electrode coupled to the stimulation circuit, the at least one electrode providing brain stimulation to targeted areas of the brain.

17. The system of claim 1, wherein the at least one electrode provides brain stimulation to targeted areas of the brain in response to signals received from the stimulation circuit.

18. A method of providing brain stimulation, comprising:
harvesting power in a power harvesting circuit in a device implantable under the scalp of a patient; and
providing brain stimulation to targeted areas of the brain with at least one electrode connected to the power harvesting circuit.

19. The method of claim 18, wherein the power harvesting circuit harvests radio frequency energy.

20. The method of claim 18, wherein the power harvesting circuit harvests energy by an inductively coupled power circuit in the device.

21. The method of claim 18, wherein the power harvesting circuit harvests energy by a non-inductively coupled power circuit in the device.

22. The method of claim 18, wherein the brain stimulation is used to treat Parkinson's disease.

23. The method of claim 18, wherein the brain stimulation is used to treat stroke patients.
